(19) United States
(12) Patent Application Publication
(10) Pub. No.: US 2024/0374141 A1
(43) Pub. Date: Nov. 14, 2024

(54) DENTAL COIL AND MAGNETIC RESONANCE DEVICE FOR IMAGING A JAW REGION

(71) Applicants: Siemens Healthineers AG, Forchheim (DE); Dentsply Sirona Inc., York, PA (US); SIRONA Dental Systems GmbH, Bensheim (DE)

(72) Inventors: Andreas GREISER, Erlangen (DE); Edgar MUELLER, Heroldsbach (DE); Lars LAUER, Neunkirchen (DE); Stephan BIBER, Erlangen (DE); Mario ZELLER, Erlangen (DE); Matthias GEBHARDT, Erlangen (DE); Johannes ULRICI, Darmstadt (DE); Stefan WUNDRAK, Bensheim (DE); Titus LANZ, Wuerzburg (DE); Florian ODOJ, Veitshoechheim (DE); Tobias WICHMANN, Rimpar (DE); Joerg ROTHARD, Litzendorf (DE); Sebastian DENNERT, Lonnerstadt (DE); Marco GEISSNER, Estenfeld (DE)

(73) Assignees: Siemens Healthineers AG, Forchheim (DE); Dentsply Sirona Inc., York, PA (US); SIRONA Dental Systems GmbH, Bensheim (DE)

(21) Appl. No.: 18/692,594

(22) PCT Filed: Aug. 22, 2022
(86) PCT No.: PCT/EP2022/073273

§ 371 Date: Mar. 15, 2024

(30) Foreign Application Priority Data
Sep. 17, 2021 (DE) 102021210304.9

(51) Int. Cl. A61B 5/00; A61B 5/055; G01R 33/34

(52) U.S. Cl. CPC A61B 5/004; A61B 5/055; G01R 33/34084

(57) ABSTRACT

A dental coil comprises a first element and a second element. The first element is composed of a dimensionally stable material and has a recess that is configured to receive at least one of a mouth region or a nose region of a patient when the dental coil is positioned on the jaw region of the patient during use. The second element has a flexible element that is configured to allow the flexible element to take the shape of the jaw region of the patient. The first element and the second element have an antenna configured to receive high-frequency signals in a frequency and power range of a magnetic resonance measurement.

### CROSS-REFERENCE TO RELATED APPLICATIONS

This application is the National Phase under 35 U.S.C. § 371 of PCT International Application No. PCT/EP2022/073273, which has an international filing date of Aug. 22, 2022, which designated the United States of America, and which claims priority to German Patent Application No. 10 2021 210 304.9, filed Sep. 17, 2021, the entire contents of each of which are hereby incorporated herein by reference.

### FIELD

The present invention relates to a dental coil which is designed to receive high-frequency signals in a frequency and power range of a magnetic resonance measurement of a jaw region of a patient. The present invention additionally relates to a magnetic resonance device comprising a dental coil, said magnetic resonance device being designed to perform an imaging scan on a jaw region of a patient.

### BACKGROUND

Diseases of the teeth and the periodontium, such as e.g. caries or periodontitis, are typically diagnosed currently using X-ray-based imaging methods. Conventional or digital X-ray projection methods, as well as recently also three-dimensional X-ray methods, are primarily used in this case. One example of a three-dimensional X-ray method is digital volume tomography, which can be used for imaging teeth and the viscerocranium.

A major disadvantage of X-ray methods is the need to use ionizing radiation for the imaging process. Magnetic resonance tomography is representative of an imaging method which avoids ionizing rays. This typically provides a better soft tissue contrast than X-ray methods and supports three-dimensional imaging of an examination subject as standard. Furthermore, magnetic resonance tomography enables imaging of cysts as well as detection of a degradation of dentin before this becomes detectable by means of an X-ray method. Magnetic resonance tomography therefore represents a potential alternative to known X-ray methods for the imaging of a dental region and/or a jaw region as well as for the diagnosis of dental diseases of the examination subject.

Magnetic resonance tomography is a well-known imaging modality by means of which magnetic resonance images of an internal region of the examination subject can be generated. When a magnetic resonance imaging procedure is carried out, the examination subject is normally positioned in a strong, static and homogeneous basic magnetic field (B0 magnetic field) of a magnetic resonance device. The basic magnetic field can exhibit magnetic field strengths of 0.2 tesla to 7 tesla such that nuclear spins of the examination subject align themselves along the basic magnetic field. In order to trigger so-called nuclear spin resonances, high-frequency signals, also known as radiofrequency excitation pulses (B1 magnetic field), are radiated into the examination subject. Each excitation pulse causes a magnetization of certain nuclear spins of the examination subject to deviate from the basic magnetic field by an amount that is also known as the flip angle. An excitation pulse can at the same time have a magnetic alternating field at a frequency which corresponds to the Larmor frequency for the respective static magnetic field strength. The excited nuclear spins can exhibit a rotating and decaying magnetization (nuclear spin resonance) which can be captured as a magnetic resonance signal by means of special antennas. Magnetic gradient fields can be superimposed on the basic magnetic field for spatial encoding of the nuclear spin resonances of the examination subject.

The received magnetic resonance signals are typically digitized and stored as complex values in a k-space matrix. This k-space matrix can be used as a basis for reconstructing magnetic resonance images as well as for determining spectroscopic data. A magnetic resonance image is typically reconstructed by means of a multidimensional Fourier transform of the k-space matrix.

Owing to the avoidance of ionizing radiation, magnetic resonance tomography is suitable in particular for continuous diagnostic monitoring of dental diseases and/or of dental development as part of a longitudinal imaging study. In longitudinal imaging studies, multiple imaging examinations are typically performed in order to determine a progression of a disease or a success of a therapeutic treatment over a predetermined period of time. However, diagnostically relevant areas of the jaw region of a patient, such as e.g. an oral cavity, a set of teeth, a dental arch or a tooth, provide only a small volume that is available for generating magnetic resonance signals. Furthermore, conventional volume and surface coils, such as e.g. head coils and lay-on coils, are placed at a relatively great distance from the diagnostically relevant region. However, a great distance can increase a signal-to-noise ratio of captured magnetic resonance signals and consequently reduce the quality of magnetic resonance images of the teeth of the patient reconstructed therefrom.

### SUMMARY

It is therefore an object of the present invention to provide a dental coil which enables high-quality magnetic resonance images of the jaw region of the patient to be acquired.

This object is achieved according to the present invention by means of the subject matters of the independent claims. Advantageous embodiment variants and beneficial developments are the subject matter of the dependent claims.

The dental coil according to the present invention comprises a first element, the first element consisting of a dimensionally stable material and comprising a recess that is suitable for accommodating a mouth region and/or a nose region of a patient when the dental coil is positioned in a manner appropriate for use on a jaw region of the patient.

A dimensionally stable material can denote any material which exhibits a negligible deformation when the dental coil is positioned in a manner appropriate for use on a jaw region of the patient. Preferably, the dimensionally stable material has the least possible interaction with magnetic fields, with the result that the first element is essentially undetectable for a magnetic resonance measurement. Examples of suitable dimensionally stable materials include plastics such as silicone, polyesters and polycarbonates, but also various ceramics such as aluminum oxide or zirconium oxide. In addition, other materials, such as e.g. glass or glass fiber reinforced plastics, are of course also possible. The recess of the first element can constitute a hole or an opening which allows the patient to breathe and/or provides an air supply for the mouth and/or nose region when the dental coil is positioned in a manner appropriate for use on the jaw region. The first element may be understood in particular as a frame which encloses the mouth and/or nose region of the patient and/or makes it easier to position the dental coil on the jaw region of the patient.

The dental coil according to the present invention further comprises a second element, the second element having a flexible element which is designed to allow the second element to conform to the shape of the jaw region of the patient by means of the flexible element.

A flexible element is preferably reversibly deformable. The second element may be made in a single piece from the flexible element or consist of segments which allow a reversible deformation of the flexible element. A segment of the flexible element can constitute e.g. a hinge, a joint, a bearing or the like. It is conceivable that the flexible element is reversibly deformable by means of a segment. In this case the segment can comprise an elastic and/or a dimensionally stable material.

The flexible element preferably comprises a plastically or elastically deformable material. The flexible element may also be made entirely out of a plastically or elastically deformable material. Examples of such materials are plastics such as polyethylene, polyurethane, polyamide and polyester. In addition, materials on a natural basis, such as e.g. rubber or fibrous materials, are also conceivable. In a preferred embodiment variant, the flexible element is implemented as foam material, fiber material or the like in order to achieve a low density and consequently a low weight. The flexible element may also be deformable in such a way that the second element can be shaped to conform to individual geometries of a plurality of different jaw regions of different patients. In a preferred embodiment variant, the flexible element is deformable by means of a manual action of force exerted by an operator of a magnetic resonance device or by the patient. Conforming to the shape of the jaw region of the patient by means of the flexible element may in particular include a deformation and/or a matching of a surface contour of the second element. The second element may in the process be brought into contact with a surface of the skin of the jaw region (e.g. by a downward pressing action). However, it is also conceivable that the second element is shaped to conform to the jaw region of the patient without contact with the surface of the skin.

The second element can be aligned in a position of the dental coil appropriate for use on the jaw region of the patient substantially tangentially to a cheek of the patient, in particular parallel to a row of adjacent molars of the patient. The second element can further have a curvature which is shaped to conform to a curvature of the cheek and/or of a part of the dental arch of the patient.

The dental coil according to the present invention is designed in particular to position and/or hold the first element and the second element in place simultaneously on the jaw region of the patient. This enables magnetic resonance signals of the jaw region to be recorded by means of the first element and the second element in the course of a magnetic resonance measurement of the jaw region of the patient.

The first element and the second element of the dental coil according to the present invention have an antenna which is designed to receive radiofrequency signals in a frequency and power range of a magnetic resonance measurement.

An antenna can constitute a coupling element between electromagnetic waves conducted in signal conductors and unconducted electromagnetic waves, i.e. waves located in a free space. The antenna is embodied in particular to receive electromagnetic waves in the range of a magnetic resonance frequency of a magnetic-resonance-active atomic nucleus. Electromagnetic waves relevant to magnetic resonance measurements can be radiofrequency signals or magnetic resonance signals comprising frequencies between 1 and 500 MHZ, preferably between 10 and 300 MHz. The magnetic resonance signals of typical atomic nuclei to be examined can exhibit a low power of a few microwatts up to several milliwatts.

A signal conductor is preferably an electrically conducting wire. The wire of the signal conductor can have an oval or polygonal cross-section and be suitable for continuously transmitting the above-stated power levels. It is also conceivable that the signal conductor is implemented as a conductor track on a printed circuit board and has an approximately rectangular cross-section. The signal conductor can consist of copper. However, other electrically conducting metals, such as e.g. gold or aluminum, are also conceivable.

The antenna of the dental coil according to the present invention preferably includes a contact protection means to protect an examination subject against voltages and/or burns. For this purpose, the signal conductor of the antenna can have e.g. a coating and/or a sheathing made of plastic. Suitable plastics include e.g. polytetrafluorethylene (PTFE) or various polysiloxanes. When a magnetic resonance signal is received, a current can be induced in the signal conductor and transmitted by means of a suitable electric connection cable to a magnetic resonance device. Such an electric connection cable can be e.g. a coaxial cable having a shielding in order to avoid electromagnetic interference from an environment.

It is conceivable that the dental coil comprises an electronic circuit which is connected to the antenna. The electronic circuit can comprise one electronic component or a combination of a plurality of electronic components, such as e.g. transistors, resistors, capacitors, diodes, conductor tracks and the like. The electronic circuit may in particular have a protective circuit which is suitable for protecting the antenna against an overload. In order to avoid magnetic attraction forces, standing waves, heat buildup and comparable undesirable effects, the electronic circuit may include a high proportion of non-magnetic materials as well as corresponding standing wave traps and/or baluns. The electronic circuit preferably has a printed circuit board (PCB) or a comparable substrate which is suitable for receiving the electronic components in a predetermined position relative to one another.

The dental coil according to the present invention may of course comprise a plurality of antennas according to an above-described embodiment variant. In this case the antennas can be arranged spaced apart from one another, adjacent to one another or partially overlapping. Further, the antennas can be arranged in the form of a grid or matrix. For example, the first element can have a first antenna or a first number of antennas, while the second element has a second antenna or a second number of antennas.

It is furthermore conceivable that the dental coil according to the present invention has at least one antenna which is configured to transmit a radiofrequency signal in a direction of the examination subject, such as e.g. a jaw region of the patient. Depending on the basic magnetic field of a magnetic resonance device, the radiofrequency signal emitted by the at least one antenna can lie for example in a power range of a few watts up to several kilowatts. The radiofrequency signal transmitted by the at least one antenna can represent in particular a B1 magnetic field. A part of the dental coil having the at least one antenna can for example constitute a transmit unit of the dental coil.

In a preferred embodiment variant, the first element comprises a number of antennas which are designed to receive magnetic resonance signals from a jaw region of the patient. The number of antennas may be integrated and/or embedded in the first element or be carried by the latter. The dimensionally stable material of the first element can be designed to provide a contact protection means for the patient according to an above-described embodiment variant.

A recess can constitute an oval or a polygonal cutout in the first element. The recess is preferably characterized by a section of the first element that is free from signal conductors. The recess can in particular be configured to provide a free space for a nose and/or a mouth of the patient when the dental coil is positioned in a position appropriate for use for performing a magnetic resonance measurement on the jaw region of the patient. A contact between the nose and/or the mouth of the patient and the first element can thus be avoided. In a preferred embodiment variant, the antenna or antennas of the first element is or are arranged in such a way that the recess is free of signal conductors. For this purpose, the antenna or antennas can surround the recess for example in a loop shape. The first element can have one or more recesses. For example, the first element can have a first recess for the nose and a second recess for the mouth of the patient. However, it is also conceivable that the first element has a single recess which can accommodate both the nose and the mouth of the patient.

With the dental coil positioned in a manner appropriate for use on the jaw region of the patient, the first element can be aligned substantially parallel to a frontal plane of the patient. It is equally conceivable that the first element is aligned substantially parallel to a dental arch of the patient, in particular parallel to a row of incisors of the patient, when the dental coil is positioned in a manner appropriate for use. With the dental coil positioned in a manner appropriate for use, the first element can lie close to a surface of the skin of the jaw region of the patient or be disposed at a predetermined distance of less than two centimeters, less than one centimeter or less than five millimeters from the surface of the skin of the jaw region of the patient.

The second element preferably has an antenna or a number of antennas which is or are held in place by the second element. The antenna or the number of antennas can for example be integrated and/or embedded in the second element and/or be connected to the latter. Preferably, the antenna or the number of antennas is or are reversibly deformable in order to be shaped to conform to the contour of the jaw region of the patient. The second element may be embodied as a wing or a side piece at least partially enclosing a left cheek and/or a right cheek of the patient when the dental coil is positioned in a manner appropriate for use on the jaw region. The second element can have in particular a first side piece and a second side piece, the first side piece being shaped to conform to the contour of the left cheek of the patient and the second side piece being shaped to conform to the contour of the right cheek of the patient. However, it is also conceivable that the second element has only one side piece. In this case the dental coil may also have a third element which is arranged on the cheek of the patient lying opposite the second element when the dental coil is positioned in a manner appropriate for use and which is shaped to conform to the contour thereof. The third element can be implemented analogously to an embodiment variant of the second element.

In a preferred embodiment variant, the dental coil according to the present invention has a retainer which is embodied to hold and/or to position the first element and/or the second element in a position appropriate for use on the jaw region of the patient. In a simple example, the retainer can have two struts or carrier elements which flank or enclose the head of the patient on the left side and right side of the head when the dental coil is positioned in a manner appropriate for use. The retainer can further comprise a holding element which is embodied to make a fine adjustment of an orientation and/or a position of the first element and/or the second element relative to the jaw region of the patient. However, it is also conceivable that the retainer comprises a positioning unit which is embodied to perform a rough positioning of the dental coil relative to the patient and/or a patient support and positioning device. A patient support and positioning device can in this case constitute any structure which assists the patient in maintaining a predetermined position and/or body posture during a magnetic resonance measurement. The patient support and positioning device may be embodied in particular to support a body region of the patient in a sitting, lying and/or standing body posture.

By providing the first element with a recess, a restriction of the breathing of the patient during a magnetic resonance measurement of the jaw region of the patient is advantageously reduced or avoided. Also, a contact area of the dental coil on the surface of the skin of the jaw region of the patient can be reduced to an area of the first element, which contributes to an increased level of comfort for the patient and reduces the risk of the magnetic resonance measurement being prematurely terminated by the patient. Furthermore, a manual positioning of the dental coil on the jaw region of the patient can be simplified since an orientation and a position of the dental coil relative to the jaw region of the patient are already predefined by means of the recess in the first element. This advantageously enables a time saving to be achieved during the positioning of the dental coil.

By providing the second element which can be shaped to conform to the jaw region of the patient, it is advantageously possible to position one or more antennas of the dental coils at a particularly short distance from and/or individually matched to the jaw region. A signal-to-noise ratio of received magnetic resonance signals can advantageously be increased as a result.

In an embodiment variant of the dental coil according to the present invention, the second element can be positioned independently of the first element.

The dental coil according to the present invention can for example comprise a retainer and/or a holding element which are designed to hold the first element and/or the second element in the position appropriate for use on the jaw region of the patient. The first element and the second element can e.g. be mechanically connected to different sections of the retainer and/or the holding element which are reversibly deformable independently of one another. It is also conceivable that the different sections of the retainer and/or the holding element can be positioned and/or oriented relative to one another by means of an adjustment mechanism, such as e.g. a hinge, a joint or a comparable mechanism.

An ability to position the first element and the second element independently of each other can mean that the first element and the second element are present separately from each other, in particular without a mechanical connection to each other. In this case a position of the second element can be set independently of a position of the first element. It can, however, also mean that the second element is mechanically connected to the first element. In this case, in particular a shape and/or a relative position of at least a part of the second element with respect to the first element can be adjusted by means of the flexible element.

An independent positioning of the first element and the second element can simplify an adjustment of the dental coil to an individual geometry of a jaw region and/or reduce a distance between the jaw region and the dental coil. A signal-to-noise ratio of the dental coil can advantageously be increased as a result.

In an alternative embodiment variant of the dental coil according to the present invention, the first element and the second element are mechanically connected to each other, the second element being held in place by the first element.

It is conceivable that the first element and the second element are connected to each other by means of a hinge or a joint in order to improve an adjustment and/or a shaping of the dental coil to conform to the contour of the jaw region of the patient. However, it is also conceivable that the possibility to adjust and/or shape the dental coil to conform to the contour of the jaw region of the patient is already given by means of the flexible element. A mechanical connection between the first element and the second element can be realized by means of any material-to-material bonded, force-fitting and/or positive-locking connection. A third element can of course also be mechanically connected to the first element or be positioned independently of the first element.

The dental coil can be positioned particularly easily on the jaw region of the patient by means of a mechanical connection of the first element to the second element. The time required for preparing the magnetic resonance measurement of the jaw region can advantageously be reduced as a result.

In a further embodiment variant, the dental coil according to the present invention additionally comprises an adjustment mechanism which is designed to adapt the shape of the flexible element in order that the second element conforms to the contour of the jaw region of the patient.

In an embodiment variant, the second element can be shaped by means of the adjustment mechanism to conform to the external geometry of the jaw region of the patient. For this purpose, the adjustment mechanism can comprise a guide mechanism, a clamping mechanism, a clasping mechanism, a bending mechanism, a pulling mechanism or the like which is configured to deform the second element in the position appropriate for use on the jaw region of the patient and/or to set a bending radius of the second element in accordance with a contour of the jaw region of the patient. It is conceivable that the adjustment mechanism is mechanically connected to the retainer of the dental coil.

The adjustment mechanism preferably comprises an actuating element which is designed to transmit a force onto the second element. An actuating element can for example be a screw, a pin, a bolt and/or an elastic element which is mounted so as to be positionable and/or can be clamped in place between the retainer and the second element by means of the adjustment mechanism. In this case a force exerted onto the adjustment mechanism by means of the actuating element can be transmitted onto the second element so that the second element or a section of the second element is positioned relative to the jaw region of the patient. The adjustment mechanism may in particular comprise a plurality of actuating elements which are designed to adapt a surface contour of a side of the second element facing toward the jaw region to conform to a surface contour of the jaw region of the patient. In this case the second element can come into contact with the surface of the skin of the jaw region of the patient. However, it is also conceivable that a predetermined distance of less than two centimeters, less than one centimeter or less than five millimeters is maintained between the second element and the surface of the skin of the jaw region of the patient.

The actuating element can further comprise an elastic element, such as e.g. a synthetic or natural elastomer, a spring, a foam material or the like. When the dental coil is positioned in a manner appropriate for use on the jaw region, the elastic element can be clamped in place between the second element and the positioning unit and/or the retainer and can transmit an elastic restoring force onto the second element. The second element is preferably deflected by means of the elastic restoring forces of the elastic element in the direction of the jaw region of the patient and in the process is shaped to conform to the jaw region of the patient. However, the elastic element can also represent a holding element which holds at least the second element, but also the first element and/or the third element, in place in a position appropriate for use on the jaw region of the patient.

An advantage of the adjustment mechanism according to the present invention represents a particularly simple and/or time-efficient reduction of the distance between one or more antennas of the dental coil and the jaw region of the patient. An increased signal-to-noise ratio of received magnetic resonance signals and/or a reduced preparation time for a magnetic resonance measurement of the jaw region of the patients can advantageously be achieved as a result.

According to a further embodiment variant of the dental coil according to the present invention, the flexible element comprises an intelligent (smart) material which is embodied to modify a material property as a function of a predetermined excitation signal, the adjustment mechanism being embodied to assist the matching of the shape of the second element by means of a transmission of the excitation signal to the flexible element.

A smart material can designate any material in which a material property of the second element is changed in a predetermined manner by means of a suitable excitation signal. In this case a material property can relate in particular to a deformability of the smart material. Further examples of affected material properties of the smart material include inter alia a cohesive force, an elasticity, a rigidity, a ductility or a strength. A material property of the smart material can preferably be set by means of the excitation signal in such a way that the second element is deformable with respect to an unexcited reference state when a reduced force is applied. However, it is also conceivable that the smart material of the second element is flexibly deformable in the unexcited reference state when force is exerted manually, whereas the smart material exhibits a reduced flexibility and/or deformability during and/or after application of the excitation signal (excited state). Furthermore, in the unexcited reference state, the smart material may also exhibit an increased rigidity, which is reduced during and/or after the application of the excitation signal.

An excitation signal can comprise any electrical, thermal and/or electromagnetic signal. The excitation signal can further comprise a mechanical vibration and/or a chemical reaction. In one example, the smart material comprises a shape-memory polymer and/or a shape-memory alloy which assume a predetermined shape if a predetermined temperature is exceeded and/or when irradiated with electromagnetic radiation of a predetermined wavelength (e.g. radiofrequency signals of a magnetic resonance device, light of a predetermined spectrum, etc.). In a further example, the smart material is embodied to melt when subjected to a small increase in temperature (thermal excitation signal), such as e.g. upon contact with the surface of the skin of the jaw region, and to enable an improved shaping of the second element to conform to the jaw region. It is furthermore conceivable that the smart material is deformed due to a piezo effect or inverse piezo effect when an electrical voltage and/or an electric current are/is applied (cf. piezoelectrics). In addition, the second element may also comprise an electrorheological and/or magnetorheological fluid, an electrostrictive and/or magnetostrictive material and/or a functional polymer. Furthermore, micro- or nanoencapsulated materials and carbon nanotubes also come into consideration as smart materials.

The time and effort required in order to shape or contour the second element to conform to the jaw region of the patient, in particular of a plurality of different patients, can advantageously be reduced thanks to the use of smart materials. As a result, the antenna of the second element can be positioned in a reproducible and efficient manner at a short distance from the jaw region of the patient even in the event of an increase in patient population.

According to a further embodiment variant, the dental coil according to the present invention further comprises a retainer which is mechanically connected to the first element and/or the second element.

The retainer is preferably mechanically connected to the first element and the second element. The retainer can be implemented according to an above-described embodiment variant. For example, the retainer has two carrier elements which flank the head of the patient on two sides when the dental coil is positioned in a manner appropriate for use.

The first element and/or the second element can be positioned relative to the patient by means of the retainer, the retainer being designed to hold the first element and/or the second element in place in the position appropriate for use on the jaw region of the patient.

According to an above-described embodiment variant, the retainer can be positioned relative to the patient and/or the patient support and positioning device by means of the positioning unit. In an embodiment variant, the retainer has a holding element which is mechanically connected to one or more carrier elements of the retainer as well as to the first element and/or the second element. It is conceivable that the retainer has a mechanism which is embodied to set a position and/or an orientation of the holding element or of the first element and/or the second element relative to the patient and/or the patient support and positioning device. The mechanism can be implemented according to an above-described embodiment variant, e.g. as a joint, a hinge, but also a pivot bearing, a roller bearing, a slide bearing or the like. In particular, the retainer provides a necessary mechanical stability in order to position and hold the first element and/or the second element in place in a manner appropriate for use on the jaw region of the patient.

By means of the retainer according to the present invention, the dental coil can be positioned in a robust and/or reproducible manner on the jaw region of the patient. As a result, a relative movement between the dental coil and the patient during a magnetic resonance measurement of the jaw region can advantageously be avoided. This can be important in particular when the first element and/or the second element are positioned at a short distance from the surface of the skin of the jaw region of the patient in order to avoid compromising the comfort of the patient by placing the dental coil on the surface of the skin.

According to a further embodiment variant, the dental coil according to the present invention has a positioning unit which is embodied to set a position and/or an orientation of the retainer relative to the patient and/or to a patient support and positioning device.

The positioning unit is preferably embodied as a part of the retainer. The positioning unit is designed to position the retainer with the dental coil, in particular the first element and/or the second element, relative to the jaw region of the patient. The positioning unit can comprise any mechanism which is configured to adjust a position and/or an orientation of the dental coil, but also of the first element and/or the second element, relative to the patient. An adjustment of a position and/or an orientation of the dental coil can include e.g. a transporting of the dental coil along at least one spatial direction and/or an alignment of the dental coil along at least one axis of rotation. The positioning unit is preferably embodied to move the dental coil along a plurality of spatial directions and/or axes of rotation. The positioning unit can be mechanically connected to at least one part of the retainer.

In an embodiment variant, the positioning unit is designed to transport the dental coil relative to a patient support and positioning device. In this case the positioning unit and/or the retainer can have at least one first surface which is mounted immovably at a distance from a patient support surface of the patient support and positioning device. A uniform gap between the patient support and positioning device and the first surface can in this case comprise a guide mechanism or be embodied as such a mechanism. The guide mechanism can for example comprise a roller system, a rail system, a linear guide, a telescopic system or the like. The positioning unit can be embodied in particular to position the dental coil along a longitudinal axis of the patient support and positioning device by means of the guide mechanism. It is furthermore conceivable that the positioning unit has a second surface and/or a strip or a bar which is positioned at a predetermined distance from the first surface between the first surface and the patient support surface of the patient support and positioning device. A gap between the first surface and the second surface and/or the strip can in turn have a guide mechanism or be embodied as such a mechanism. The guide mechanism can be embodied to limit a movement of the retainer relative to the patient support and positioning device to at least one predetermined spatial direction and/or movement trajectory. However, it is also conceivable that the guide mechanism limits the movement of the retainer relative to the patient support and positioning device to at least two or three predetermined spatial directions and/or movement trajectories. The first surface can be embodied to receive the patient during a magnetic resonance measurement and/or to support the patient in maintaining a posture required for the magnetic resonance measurement.

In an embodiment variant, the positioning unit comprises a pivoting mechanism which is embodied to pivot or tilt the retainer relative to a longitudinal axis of the patient and/or the magnetic resonance device. For example, the first surface has a hinge or a joint which is embodied to set an angle between the dental coil and the first surface and/or the patient support and positioning device. The dental coil can preferably be tilted by means of the hinge or the joint relative to the first surface and/or the patient support and positioning device. The hinge or the joint can further comprise a mechanism which is embodied to lock the dental coil at a predetermined angle to the first surface. It is conceivable that locking the dental coil at the predetermined angle to the first surface is implemented by means of a latching mechanism, a guide mechanism, a plug-in mechanism or a clamping mechanism. In addition, any further force-fitting and/or positive-fitting locking mechanisms are of course also conceivable. In a further embodiment variant, the positioning unit is pivotably mounted by means of the pivoting mechanism, an angle of the positioning unit being settable relative to a magnetic resonance device and/or a patient.

By providing a positioning unit according to the present invention it is possible to change a position and/or an orientation of the dental coil relative to the patient in a particularly time-efficient manner. Furthermore, a blocking of an access of the patient to the patient support surface can advantageously be avoided by means of a positionable dental coil.

In a preferred embodiment variant of the dental coil according to the present invention, the retainer has a pivoting mechanism, the first element and/or the second element being mounted so as to be pivotable by a maximum angle relative to the patient by means of the pivoting mechanism when the dental coil is positioned in a manner appropriate for use on the jaw region of the patient and the maximum angle of the first element and/or the second element relative to the patient being chosen such that a head of the patient can be positioned unobstructed in a position appropriate for use relative to the retainer.

The pivoting mechanism may comprise for example a hinge, a joint, a pivot bearing, a slide bearing, an antifriction bearing, a roller bearing and/or any other mechanism which is embodied to pivot the first element and/or the second element at an angle relative to the retainer and/or the patient support and positioning device. When the dental coil is positioned in a manner appropriate for use, the first element and/or the second element can preferably be pivoted along a sagittal plane of the patient. It is however also conceivable that the first element and/or the second element can be pivoted approximately parallel to a frontal plane of the patient. During the pivoting movement, the dental coil can follow a segment of an arc that is determined by the pivoting mechanism. It is conceivable that the pivoting mechanism is incorporated in the positioning unit and/or integrated into the same according to an above-described embodiment variant.

In a preferred embodiment variant, the pivoting mechanism has a detent or resistance element which marks when a predetermined angle is reached and/or prevents an unwanted or uncontrolled pivoting movement. It is further conceivable that the pivoting mechanism comprises an arrangement of disk cams or nipples which counter a pivoting movement of the first element and/or the second element with a resistance and prevent an uncontrolled snapping shut or snapping open of the dental coil.

Depending on the design of the magnetic resonance device and/or the patient support and positioning device, the maximum angle can range between 60° and 90°, 90° and 180° or 180° and 270°. With magnetic resonance devices in which the patient assumes a standing or sitting body posture for the magnetic resonance examination, the maximum angle preferably amounts to between 60° and 90°. However, greater values for the maximum angle are also conceivable. In conventional magnetic resonance devices comprising a patient table, the maximum angle is preferably less than 180°.

By providing a pivoting mechanism it is possible for the first element and/or the second element to be positioned in a particularly time-efficient manner at a short distance from the jaw region of the patient and to be removed again completely from an access area of the patient to the patient support and positioning device. This advantageously allows an increase in the time that may be required for positioning the dental coil in a manner appropriate for use on the jaw region to be compensated for.

In a further embodiment variant of the dental coil according to the present invention, the retainer comprises a guide mechanism having a guide axis, the guide axis being aligned substantially parallel to a line of intersection of a sagittal plane and a transverse plane on the jaw region of the patient and the guide mechanism being embodied to position the first element along the guide axis in order to set a distance between the first element and the jaw region of the patient.

It is conceivable that the guide axis is aligned parallel to the line of intersection of a sagittal plane and a transverse plane on the jaw region of the patient. However, it is also conceivable that an alignment of the guide axis is tilted by an angle, e.g. an angle between 0 and 15° or between 15 and 30°, with respect to the transverse plane. In special cases, the guide axis may also be tilted by a few degrees relative to the sagittal plane.

The guide mechanism can have a guide element, such as e.g. a cylinder or a screw. For example, the screw can engage in a thread of the first element and/or the second element. Turning the screw causes the screw to be screwed into the thread of the first element and/or the second element, which are moved in the direction of the screw as a result. For this purpose, the screw can be mounted in a predetermined position relative to the retainer such that a movement of the screw in the direction of the first element and/or the second element during a turning action is avoided. The retainer may in particular have one or more pins or bolts which engage in complementary recesses or holes in the first element and/or the second element. The pins or bolts are preferably configured to provide a guide for the first element and/or the second element along an axis of the pins or bolts when the first element and/or the second element are positioned along the guide axis by means of the guide mechanism. The pins or bolts are preferably oriented parallel to the guide axis.

It is further conceivable that the first element and/or the second element are/is mechanically connected to a holding element. The screw of the guide mechanism can accordingly engage in a thread of the holding element in order to position the first element and/or the second element along the guide axis of the guide mechanism. The holding element may further have one or more recesses or holes which are embodied as complementary to the pins or bolts of the retainer. Analogously to the above-described example, the recesses or holes can provide a guide for the pins or bolts which avoids a twisting or tilting of the holding element in relation to the guide axis.

The guide axis can in particular represent an axis of rotation of the screw of the guide mechanism. The guide axis is preferably aligned parallel to a sagittal plane, in particular a median sagittal plane or mid-sagittal plane, of the patient.

A fine adjustment of the position of the first element and/or the second element on the jaw region of the patient can be made by means of the guide mechanism according to the present invention. This enables a particularly short distance to be provided between the surface of the skin of the jaw region and the first element and/or the second element, as a result of which the signal-to-noise ratio of the dental coil can advantageously be improved. It is also conceivable that the first element and the second element (but also the third element) can be positioned along the guide axis particularly uniformly on the surface of the skin of the jaw region of the patient by means of the guide mechanism. In particular a twisting or tilting of the first element and/or the second element, which can lead to an unbalanced arrangement of antennas of the first element and the second element on the jaw region, can be avoided as a result.

In an embodiment variant, the retainer of the dental coil according to the present invention has a swan-neck mechanism or a joint hose.

A swan-neck mechanism can comprise a flexible spiral tube which can be bent in any spatial direction and is embodied to maintain a curved shape. For this purpose, the swan-neck mechanism can have a screw-shaped internal wire which is wrapped in a wire having a substantially triangular cross-section.

A joint hose can be composed e.g. of formed parts like ball-and-socket joints. The formed parts similar to ball-and-socket joints can be locked together and have a predetermined holding force which exceeds a weight force of the first element and/or the second element. The joint hose can be manually deformed or bent in order to position the first element and/or the second element (as well as the third element) and maintain its shape for the duration of a magnetic resonance measurement.

It is further conceivable that the swan-neck mechanism or the joint hose has a locking mechanism which is designed to lock or secure the swan-neck mechanism or the joint hose in place in a predetermined shape. It is also conceivable that the swan-neck mechanism or the joint hose is mechanically connected to a holding element which carries the first element and/or the second element and/or holds a predetermined relative position.

Providing a swan-neck mechanism or a joint hose enables the first element and/or the second element to be positioned in a particularly simple and time-efficient manner relative to the jaw region of the patient. Furthermore, a swan-neck mechanism and a joint hose can constitute a particularly cost-effective solution for positioning the dental coil and advantageously reduce the manufacturing costs of the dental coil. In addition, a swan-neck mechanism or a joint hose provides the dental coil with an increased degree of openness. This enables patient comfort to be improved and a risk of a premature termination of a magnetic resonance measurement to be minimized.

According to a further embodiment variant of the dental coil according to the present invention, the pivoting mechanism further comprises a damping element which is embodied to dampen and/or limit a movement of the first element and/or the second element when the first element and/or the second element are/is pivoted along a predetermined path of travel.

In a preferred embodiment variant, the damping element is implemented as a mechanical spring, a pneumatic element such as e.g. a gas compression spring, or an elastic element. The damping element can in this case be aligned along the path of travel of the first element and/or the second element. For example, the spring can be positioned so that a movement of the first element and/or the second element is braked and/or dampened all the more strongly, the further the first element and/or the second element are/is pivoted along the predetermined path of travel in the direction of the jaw region of the patient.

However, it is also conceivable that the damping element comprises an arrangement of cam disks, latching elements or nipples according to an above-described embodiment variant which are embodied to counter a movement of the first element and/or the second element along the predetermined path of travel with a resistance. The damping element can be further embodied as a braking element which brakes or limits a movement of the first element and/or the second element by means of a force-fitting connection to the retainer. A resistance which is opposed to the movement of the first element and/or the second element along the predetermined path of travel by means of the damping element can in this case vary with a distance between the first element and/or the second element and the jaw region and increase in the direction of the jaw region of the patient.

By providing a damping element according to the present invention it is advantageously possible to avoid or limit an uncontrolled folding shut or pivoting of the dental coil. In particular in the case of magnetic resonance devices in which the patient is aligned in a vertical or standing position, this can prevent an injury to the patient as a result of an improper positioning of the dental coil by an operator or the patient.

In a further embodiment variant, the dental coil according to the present invention further comprises a flexible holding element which is connected at least to the second element when the dental coil is positioned in a manner appropriate for use on the jaw region of the patient, the adjustment mechanism being embodied as a pull mechanism which is mechanically connected to the flexible holding element and is configured to provide a tractive force which guides the flexible holding element in a direction of the jaw region of the patient.

The first element and/or the second element can be connected to the flexible holding element and/or be held in place by the latter. The first element and/or the second element can be connected in particular in a force-fitting, positive-locking and/or material-to-material bonded manner to the flexible holding element. In a preferred embodiment variant, the flexible holding element can be positioned relative to a carrier element and/or a positioning unit of the retainer and holds at least the second element in place in a position appropriate for use on the jaw region of the patient. The pull mechanism can in particular constitute an adjustment mechanism according to an above-described embodiment variant.

It is in particular conceivable that the flexible holding element is positioned between the jaw region of the patient and the retainer when the dental coil is positioned in a manner appropriate for use. The flexible holding element can in this case have a substantially cuboidal or homoeomorphic shape with respect to a cuboid which circumferentially encloses the jaw region from a left cheek region up to a right cheek region of the patient. Preferably, a first end of the flexible holding element on the left cheek region and a second end of the flexible holding element on the right cheek region are mechanically connected to the pull mechanism. The pull mechanism can have a pulling element, such as e.g. a strap, a rope, a net, a cord or the like, which guides or deflects the first end and the second end by means of the pull mechanism in a direction facing away from the patient. Such a direction can point substantially from a dorsal side of the patient in a direction facing away from the patient. In this case the pull mechanism can comprise any mechanism which is embodied to exert a tractive force on the pulling element.

In an embodiment variant, the flexible holding element can be shaped by means of the pull mechanism to conform to the jaw region of the patient. This can mean that the flexible holding element can be positioned along the jaw region of the patient by means of the pull mechanism. The flexible holding element can for example rest on the surface of the skin of the patient and be shaped to conform to said skin surface by means of the tractive force. However, it is also conceivable that the flexible holding element is spaced at a distance from the surface of the skin of the patient by means of the first element and/or the second element. In this case the flexible holding element can be embodied to elastically deform at least the second element when a tractive force is exerted onto the pulling element and to be shaped to conform to the contour of the jaw region of the patient.

In an alternative embodiment variant, the flexible holding element is embodied to be positioned relative to the retainer and inserted into the retainer under elastic deformation. In this case the flexible holding element can shape the second element to conform to the jaw region of the patient independently of the adjustment mechanism. For example, elastic restoring forces of the flexible holding element can be chosen such that the flexible holding element forms a force-fitting connection to the retainer. It is in particular conceivable that, when positioned in a manner appropriate for use, the flexible holding element is positioned and/or inserted between the jaw region of the patient and the retainer. In this case the flexible holding element can be shaped to conform to the jaw region of the patient. The elastic restoring force of the flexible holding element can in particular be geared to a predetermined group of patients, such as e.g. children, adolescents, adults or seniors, such that, when positioned in a manner appropriate for use on the jaw region of the patient, the flexible holding element exerts a predetermined pressure onto the jaw region of the patient and/or the second element. As described above, at least the second element can be positioned between the surface of the skin of the jaw region of the patient and the flexible holding element and be shaped to conform to the jaw region of the patient when the flexible holding element is positioned.

By providing a flexible holding element according to the present invention it is advantageously possible for the second element to be shaped to conform to the contour of the jaw region of the patient in a particularly time-efficient manner.

In an embodiment variant, the local coil according to the present invention further comprises a third element having a flexible element, wherein the flexible element is embodied to allow the third element to be shaped to conform to the contour of the jaw region of the patient and wherein the third element is arranged on an opposite side of the jaw region of the patient to the second element when the dental coil is positioned in a manner appropriate for use, wherein the third element has an antenna which is embodied to receive radiofrequency signals in a frequency and power range of a magnetic resonance measurement.

The third element can be designed according to an above-described embodiment variant. The third element can be designed in particular as a part of the second element, be mechanically connected to the second element or be present separately from the second element. The third element shares the advantages of the second element. In particular, the shaping to the geometry of the jaw region by means of the dental coil can be further improved by means of a separate third element.

In a further embodiment variant, the adjustment mechanism is embodied to position the first element and the second element relative to one another.

For this purpose, the first element and/or the second element can be mechanically connected to separate carrier elements of the retainer. The separate carrier elements can comprise a reversibly deformable material and/or an adjustment mechanism which are embodied to position the second element relative to the first element. In an analogous manner, the third element can be positionable by means of a separate carrier element of the retainer relative to the second element.

However, it is also conceivable that the first element and the second element are mechanically connected to a holding element of the retainer, such as e.g. to the flexible holding element. The holding element can in this case comprise a plastically or elastically deformable material. When the holding element is being deformed, the first element can accordingly be positioned relatively with respect to the second element.

By providing a retainer which is embodied to position the first element, the second element and/or the third element relative to one another, it is advantageously possible to shape the antenna or antennas of the dental coil to conform to an individual geometry of the jaw region of the patient.

According to an embodiment variant, the dental coil according to the present invention comprises a fixing element which is designed to connect the retainer and/or the first element mechanically to the head of the patient in order to fix the head of the patient in place in a predetermined position relative to the first element.

The fixing element can be embodied for example as a loop-shaped cord, a strap or the like which is mechanically connected to the retainer and/or the first element and encompasses a chin, a lower jaw and/or the back part of the head of the patient. The cord or the strap can be embodied in particular to secure the first element and/or the retainer in place on the head of the patient and/or to brace them against the chin or the lower jaw. In an embodiment variant, the fixing element can be connected to a pull mechanism according to an above-described embodiment variant which is configured to apply a predetermined tractive force to the fixing element such that the first element and/or the retainer are/is pressed onto the surface of the skin of the jaw region of the patient with a force equivalent to the predetermined tractive force.

In a further embodiment variant, the fixing element can comprise an elastic material, such as e.g. an elastomer, in particular a rubber band or an elastic foam material. For example, as described above, the fixing element can be elastically deformed during a positioning between the retainer and/or the first element and the jaw region of the patient, elastic restoring forces of the fixing element fixing the jaw region and/or the head of the patient in place relative to the retainer and/or the first element. In particular, the fixing element can be embodied as an elastic foam material. The elastic foam material can also be clamped between the retainer and the second element and can press the second element onto the surface of the skin of the patient so that the head of the patient is fixed in place in a predetermined position relative to the first element.

In a further embodiment variant, the fixing element is mechanically connected to the first element and can be positioned by means of a suitable mechanism (e.g. the guide mechanism) on the surface of the skin of the jaw region of the patient. In this case the fixing element may comprise in particular an elastic material, such as e.g. a cushion filled with fluid (e.g. air, inert gas or a liquid), a foam material, an elastomer or the like, which is positioned on a side of the first element facing toward the jaw region of the patient. The fixing element can in particular be configured to distribute and/or cushion a pressure exerted onto the surface of the skin of the jaw region of the patient. The first element can preferably be positioned on the surface of the skin of the patient by means of a guide mechanism according to an above-described embodiment variant.

In an embodiment variant, the fixing element is designed to be braced between the forehead of the patient and the retainer and to provide a force-fitting connection with the retainer to the forehead of the patient.

By fixing the retainer in place on the forehead of the patient it is advantageously possible to provide a robust and reproducible positioning of the dental coil on the jaw region of the patient.

By means of the fixing element according to the present invention, the dental coil according to the present invention can advantageously be positioned in a consistent or constant position relative to the jaw region of the patient.

In a further embodiment variant, the dental coil according to the present invention further comprises a safety mechanism which is configured to transfer the dental coil into an open position when the safety mechanism is actuated by a person in order to remove the dental coil from the position appropriate for use on the jaw region of the patient.

The safety mechanism can be implemented for example as a ripcord which is embodied to remove the dental coil, the retainer, the first element and/or the second element from the position appropriate for use on the jaw region of the patient when a predetermined tractive force is exerted. Preferably, the ripcord is mechanically connected to the first element and/or the second element. However, it is also conceivable that the ripcord is mechanically connected to a retaining element and/or to the holding element which connects the first element and/or the second element to the retainer in a predetermined relative position. The first element and/or the second element can be released or disengaged from the retainer by means of a pull on the ripcord in order to release the retaining element and/or the holding element.

However, it is also conceivable that the safety mechanism comprises an electronic mechanism and an actuating element. The electronic mechanism can comprise a switch, a pushbutton, a camera and/or a microphone which are embodied to record a switching command, a gesture and/or a voice message of the patient and/or the operator. The gesture and/or the voice message can in particular encode a command of the patient or operator to transfer the dental coil to the open position. The safety mechanism may further comprise a computing unit, a control unit and/or a logic unit which output a control signal for positioning the actuating element in accordance with the switching command, the gesture and/or the voice message. The control signal can in particular be transmitted to a drive which is embodied to change a position of the actuating element. A suitable drive can be in particular an electric drive, but also a pneumatic, hydraulic, motor or manual drive. The actuating element is preferably mechanically connected to the retaining element and/or the holding element which connects the first element and/or the second element to the retainer in the predetermined relative position. In this way the safety mechanism can release the first element and/or the second element from the retainer in accordance with the switching command, the gesture and/or the voice message and transfer the dental coil into an open position.

By providing a safety mechanism according to the present invention it is advantageously possible to transfer the dental coil to an open position if the patient feels unwell or experiences discomfort and/or if a tolerable pressure onto the surface of the skin of the jaw region is exceeded. This enables accidents in the positioning of the dental coil according to the present invention to be avoided and can advantageously increase a sense of safety or confidence on the part of the patient during a magnetic resonance measurement by means of the dental coil.

According to an embodiment variant, the dental coil according to the present invention comprises a transmit unit and a receive unit, the transmit unit having at least one antenna which is configured to emit radiofrequency signals in a frequency and power range of a magnetic resonance device into a jaw region of a patient and the receive unit having at least one antenna which is embodied to receive magnetic resonance signals from the jaw region of the patient. It is conceivable that the at least one antenna of the transmit unit coincides with the at least one antenna of the receive unit.

Providing a dental coil with a transmit unit enables the at least one antenna to be positioned in immediate proximity to the jaw region of the patient. This advantageously allows an excitation of a tissue of the patient to be confined to the jaw region, thereby enabling the efficiency of the dental coil to be increased.

The magnetic resonance device according to the present invention comprises a dental coil according to an above-described embodiment variant, the magnetic resonance device being embodied to record radiofrequency signals in a frequency and power range of a magnetic resonance measurement from a jaw region of a patient by means of the dental coil in order to perform an imaging scan of the jaw region of the patient.

The magnetic resonance device can comprise a retainer according to an above-described embodiment variant. The retainer can be embodied to hold and/or position at least one component of the dental coil, such as e.g. a first element, a second element, a third element and/or a holding element, in a predetermined position relative to a jaw region of the patient. The retainer can further be mechanically connected to the magnetic resonance device, in particular to a patient support and positioning device and/or a patient table. However, it is also conceivable that the retainer is a separate component from the magnetic resonance device. The retainer can in this case be mounted on a wall and/or a ceiling of an examination room of the magnetic resonance device or be reversibly attached to the patient table. The retainer preferably has a positioning unit which is embodied to set a position of the retainer and/or the at least one component of the dental coil relative to the patient support and positioning device and/or the patient. It is further conceivable that the positioning unit is configured to set a spatial position and/or an orientation of the at least one component of the dental coil and/or the retainer.

The magnetic resonance device according to the present invention further comprises at least one electric connection cable which is configured to connect an antenna of the dental coil electrically to the magnetic resonance device. In an embodiment variant, the dental coil comprises one or more antennas which are embodied as a transmit unit. The transmit unit can be connected to a radiofrequency unit of the magnetic resonance device by means of an electric connection cable. It is conceivable that the radiofrequency unit provides an alternating current which is transmitted as a radiofrequency signal from the transmit unit into a volume of the jaw region of the patient, with the result that a B1 magnetic field is generated. In a further embodiment variant, the dental coil comprises one or more antennas which are embodied as a receive unit. The receive unit can be connected to a receiver channel of the magnetic resonance device by means of an electric connection cable. The magnetic resonance device is thus able to receive magnetic resonance signals from the jaw region of the patient and to reconstruct magnetic resonance images in accordance with the received magnetic resonance signals.

By means of the magnetic resonance device according to the present invention it is advantageously possible to enable a time-efficient and repeatable acquisition of magnetic resonance images of the jaw region of the patient. Furthermore, the magnetic resonance device according to the present invention shares the advantages of the dental coil according to the present invention according to an above-described embodiment variant.

### BRIEF DESCRIPTION OF THE DRAWINGS

Further advantages and details will become apparent from the following description of exemplary embodiments taken in conjunction with the schematic drawings, in which:

FIG. 1 shows a schematic representation of an embodiment variant of a magnetic resonance device according to the present invention,

FIG. 2 shows a schematic representation of an embodiment variant of a dental coil according to the present invention,

FIG. 3 shows a possible embodiment variant of a dental coil according to the present invention,

FIG. 4 shows a possible embodiment variant of a dental coil according to the present invention,

FIG. 5 shows a possible embodiment variant of a dental coil according to the present invention,

FIG. 6 shows a possible embodiment variant of a dental coil according to the present invention,

FIG. 7 shows a possible embodiment variant of a dental coil according to the present invention,

FIG. 8 shows a schematic representation of an embodiment variant of a dental coil according to the present invention,

FIG. 9 shows a possible embodiment variant of a retainer of a dental coil according to the present invention.

FIG. 10 shows a possible embodiment variant of a retainer of a dental coil according to the present invention,

FIG. 11 shows a possible embodiment variant of a dental coil according to the present invention.

### DETAILED DESCRIPTION

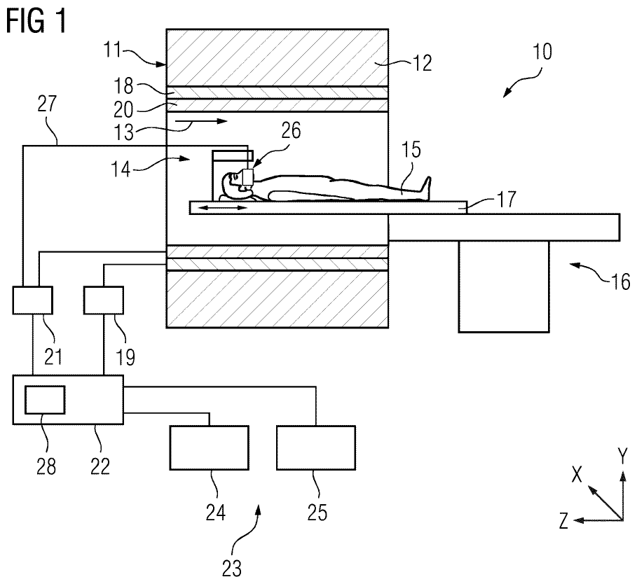

FIG. 1 schematically shows a possible embodiment variant of a magnetic resonance device 10 according to the present invention comprising a dental coil 26 according to the present invention. The magnetic resonance device 10 comprises a magnet unit 11 which has e.g. a permanent magnet, an electromagnet or a superconducting main magnet 12 for generating a strong and in particular homogeneous basic magnetic field 13 (BO magnetic field). The magnetic resonance device 10 further comprises a patient receiving zone 14 for accommodating a patient 15. In the present exemplary embodiment, the patient receiving zone 14 is embodied in the shape of a cylinder and is surrounded by the magnet unit 11 in a circumferential direction. Basically, however, embodiments of the patient receiving zone 14 differing from this example are also conceivable.

The patient 15 can be positioned in the patient receiving zone 14 by means of a patient support and positioning device 16 of the magnetic resonance device 10. For this purpose, the patient support and positioning device 16 has a patient table 17 which is embodied as movable inside the patient receiving zone 14. The magnet unit 11 additionally has a gradient coil 18 for generating magnetic gradient fields which is used for spatial encoding during a magnetic resonance measurement. The gradient coil 18 is controlled by means of a gradient control unit 19 of the magnetic resonance device 10. The magnet unit 11 may further comprise a radiofrequency antenna which in the present exemplary embodiment is embodied as a bodycoil 20 permanently integrated in the magnetic resonance device 10. The bodycoil 20 is designed for the purpose of exciting atomic nuclei located in the basic magnetic field 13 generated by the main magnet 12. The bodycoil 20 is driven by a radiofrequency unit 21 of the magnetic resonance device 10 and radiates radiofrequency signals into an examination space substantially formed by a patient receiving zone 14 of the magnetic resonance device 10. The bodycoil 20 may furthermore be embodied also to receive magnetic resonance signals.

The magnetic resonance device 10 comprises a control unit 22 for controlling the main magnet 12, the gradient control unit 19 and the radiofrequency unit 21. The control unit 22 is embodied for controlling the execution of a sequence such as e.g. an imaging gradient echo sequence, a TSE sequence or a UTE sequence. The control unit 22 further comprises an evaluation unit 28 for evaluating digitized magnetic resonance signals recorded during the magnetic resonance measurement.

The magnetic resonance device 10 further comprises a user interface 23 which has a signal connection to the control unit 22. Control information such as, for example, imaging parameters and reconstructed magnetic resonance images can be displayed for a user on a display unit 24, for example on at least one monitor of the user interface 23. The user interface 23 also has an input unit 25 by means of which magnetic resonance imaging parameters can be entered by the user.

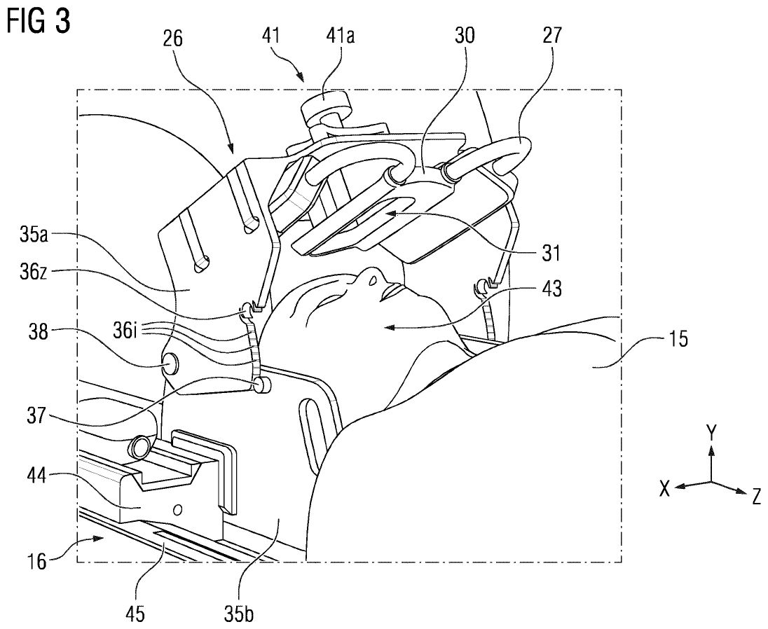

The magnetic resonance device 10 further comprises a dental coil 26 which is positioned on the jaw region 43 of a patient 15 (see FIG. 3) and transmits magnetic resonance signals from a volume of the jaw region 43 to the magnetic resonance device 10. The dental coil 26 preferably has an electric connection cable 27 which provides a signal connection to the radiofrequency unit 21 and the control unit 22. Like the bodycoil 20, the dental coil 26 can also be embodied for exciting atomic nuclei and for receiving magnetic resonance signals. A transmit unit of the dental coil 26 is driven by the radiofrequency unit 21 for the purpose of transmitting radiofrequency signals. On its outer circumference, the dental coil 26 can enclose the head of the patient 15 along a longitudinal axis of the patient 15. The transmit unit and/or a receive unit of the local coil 26 can be carried in particular by a retainer 35 and/or a holding element 33 which are adapted to conform to the external shape of the jaw region 43 of the patient 15 (see FIG. 3).

The magnetic resonance device 10 shown may of course comprise further components which are typically included in magnetic resonance devices. It is also conceivable that, instead of the cylindrical design, the magnetic resonance device 10 has magnetic-field-generating components arranged in a C-shaped, triangular or asymmetric configuration. The magnetic resonance device 10 may in particular be a dedicated magnetic resonance device 10 which is embodied to perform a magnetic resonance imaging scan of the jaw region of a standing or sitting patient 15.

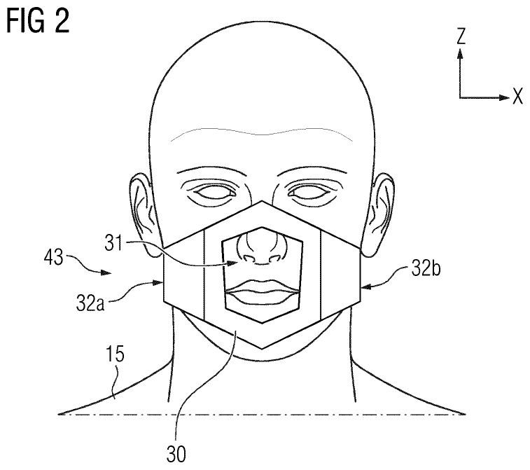

FIG. 2 shows an embodiment variant of the dental coil 26 according to the present invention in which the first element 30 and the second element 32 are positioned in the position appropriate for use on the jaw region 43 of the patient 15. In the present example, the first element 30 is mechanically connected to the second element 32, the two wings or side pieces 32a and 32b of the second element 32 being arranged at an angle to the first element 30. In this arrangement, the right wing 32a is shaped to conform to a surface contour of a right cheek region of the patient 15, while the left wing 32b is shaped to conform to the surface contour of a left cheek region of the patient 15. A wing 32a or 32b of the second element 32 can also represent a third element. The first element 30 and/or the second element 32 can be positioned at a short distance from the surface of the skin of the jaw region 43 of the patient 15 or rest against said surface.

As shown in FIG. 2, the first element 30 has at least one recess 31 which is embodied to accommodate the nose and the mouth of the patient 15. The first element 30 consists of a dimensionally stable or rigid material which is preferably shaped to conform to the mouth and/or nose region of the patient 15. In the example shown, the flexible element is made from an elastic material in which an antenna of the second element 32 is embedded. The antenna preferably has flexible or bendable signal conductors so that the second element 32 can be shaped to conform to the contour of the jaw region of the patient 15.

FIG. 3 shows an embodiment variant of the dental coil 26 according to the present invention in which an upper part of the retainer 35a of the dental coil 26 is mounted so as to be pivotable with respect to a lower part of the retainer 35b and/or a positioning unit 44 of the dental coil 26. In the example shown, the dental coil 26 has a pivoting mechanism 38 which in the present case includes a pivot bearing. The upper part of the retainer 35a with the first element 30 can accordingly be swiveled or tilted relative to the patient support and positioning device 16 and/or the patient 15 around a pivot point which is defined by a position of the pivot bearing. In the embodiment variant shown, the dental coil 26 further comprises a damping element 37 which limits or prevents an uncontrolled snapping shut of the upper part of the retainer 35a in the direction of the patient 15. The damping element 37 counters the latching elements 36i, which can be implemented for example as cam disks, nipples or convexities, with a predetermined resistance when the upper part of the retainer 35a is pivoted. The upper part of the retainer 35a further comprises a latching element 36z which terminates a movement of the upper part of the retainer 35a in the direction of the patient 15 and holds the upper part of the retainer 35a in place in a predetermined position relative to the patient 15.

The positioning unit 44 is configured to position the dental coil 26 relative to the patient support surface of the patient support and positioning device 16 and/or the patient 15. The positioning unit 44 can be positioned as shown along a suitable guide element 45. The positioning unit 44 is mechanically connected to a lower part of the retainer 35b which is connected to the upper part of the retainer 35a by means of the pivoting mechanism 38. The upper part of the retainer 35a may also be regarded as a holding element 33 for the first element 30 and/or the second element 32.

The embodiment variant of the dental coil 26 according to the present invention shown in FIG. 3 further comprises a guide mechanism 41 for making a fine adjustment to a position of the first element 30. When the dental coil 26 is positioned in a manner appropriate for use on the jaw region 43 of the patient 15, i.e. for example in a folded-shut state of the upper part of the retainer 35a, a guide axis of the screw 41a is aligned substantially parallel to a line of intersection of a sagittal plane and a transverse plane on the jaw region 43 of the patient 15.

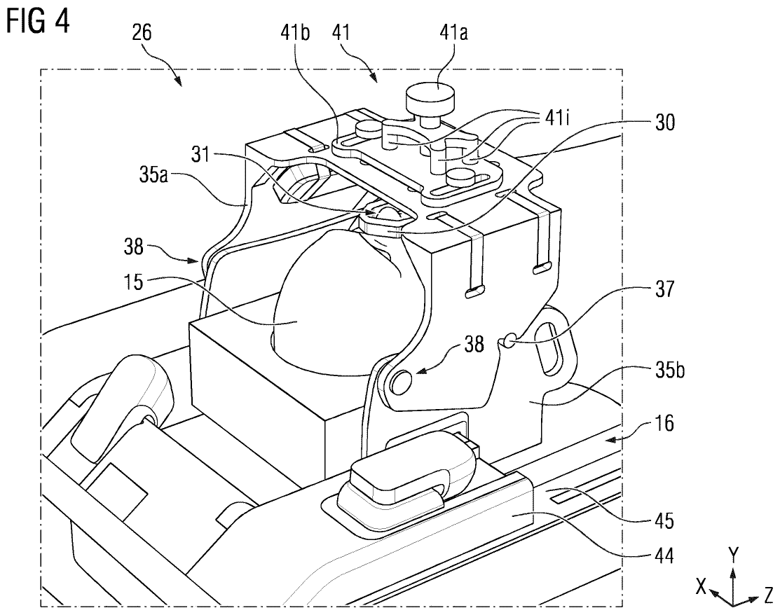

FIG. 4 shows an alternative view of the embodiment variant of the dental coil 26 according to the present invention shown in FIG. 3. The guide mechanism 41 can for example have pins or bolts 41i which pass through the upper part of the retainer 35a and are mechanically connected to the first element 30. The guide mechanism 41 is embodied to position the first element 30 along the Y-direction when the screw 41a is turned. The bolts or pins 41i of the guide mechanism 41 are in this case guided through slotted holes in the upper part of the retainer 35a and can be positioned relative to the patient 15 by means of the screw plate 41b at least along the Z-direction. The guide mechanism 41 may furthermore be interpreted also as a holding element 33 which is embodied to hold the first element 30 in place in the position appropriate for use on the jaw region 43 of the patient 15 and/or to position the first element 30 relative to the jaw region 43 of the patient 15.

FIG. 5 shows an embodiment variant of the dental coil 26 according to the present invention in which the second element 32 is shaped by means the flexible holding elements 46a and 46b to conform to an external geometry of the jaw region 43 of the patient 15. The flexible holding elements 46a and 46b can consist for example of an elastic foam material which is tailored to an expected geometry of the jaw region 43 of a patient group. Preferably, the flexible holding elements 46a and 46b are clamped or wedged between the retainer 35 and the two wings 32a and 32b of the second element 32 such that the two wings 32a and 32b are held in a position appropriate for use at a predetermined distance from the surface of the skin of the jaw region 43 of the patient 15. With a flexible holding element 46 positioned in a manner appropriate for use, the flexible element of the second element 32 is preferably deformed in such a way that a contour of one or more antennas of the second element 32 is shaped to conform to a contour of the jaw region 43 of the patient 15. The second element 32 is in this case deformed and/or positioned relative to the first element 30 by means of the flexible holding element 46.

As shown in FIG. 4, the first element 30 can in this case be positioned by means of the adjustment mechanism 41 on the surface of the skin or at a short distance from the surface of the skin of the jaw region 43 of the patient 15. The flexible holding elements 46a and 46b may also be understood as a holding element 33 which shapes the second element 32 to conform to the jaw region of the patient and holds it in place in a position appropriate for use on the jaw region.

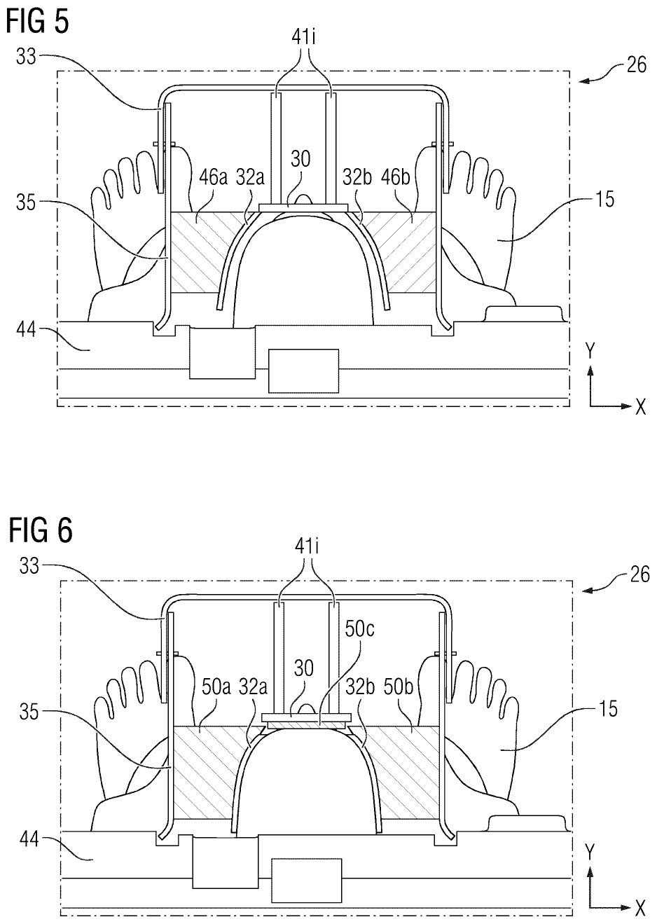

FIG. 6 shows an alternative embodiment variant of the dental coil according to the present invention in which the second element 32 is present separately from the first element 30. The wings 32a and 32b of the second element 32 can in this case be mechanically connected to a base of the retainer 35 (e.g. a first surface) or to the positioning unit 44. In this case the flexible holding elements 46a, 46b of FIG. 5 can also act as fixing elements 50a, 50b and 50c which fix the head of the patient 15 in place by pressing the two wings 32a and 32b of the second element 32 downward onto the surface of the skin of the patient 15 in a predetermined position relative to the first element 30.

In the present example, the first element 30 has a fixing element 50c which consists of an elastic material on a side facing toward a jaw region 43 of the patient 15. In this embodiment variant, the first element 30 is preferably brought into contact with the surface of the skin of the jaw region 43 of the patient 15 by means of the guide mechanism 41 so that the head of the patient 15 is fixed in place by means of the first element 30 in a predetermined position relative to the dental coil 26. The elastic material of the fixing element 50c can be embodied to distribute the pressure of the guide mechanism 41 over the jaw region 43 of the patient 15 and to increase a level of comfort of the patient 15 during a magnetic resonance measurement.

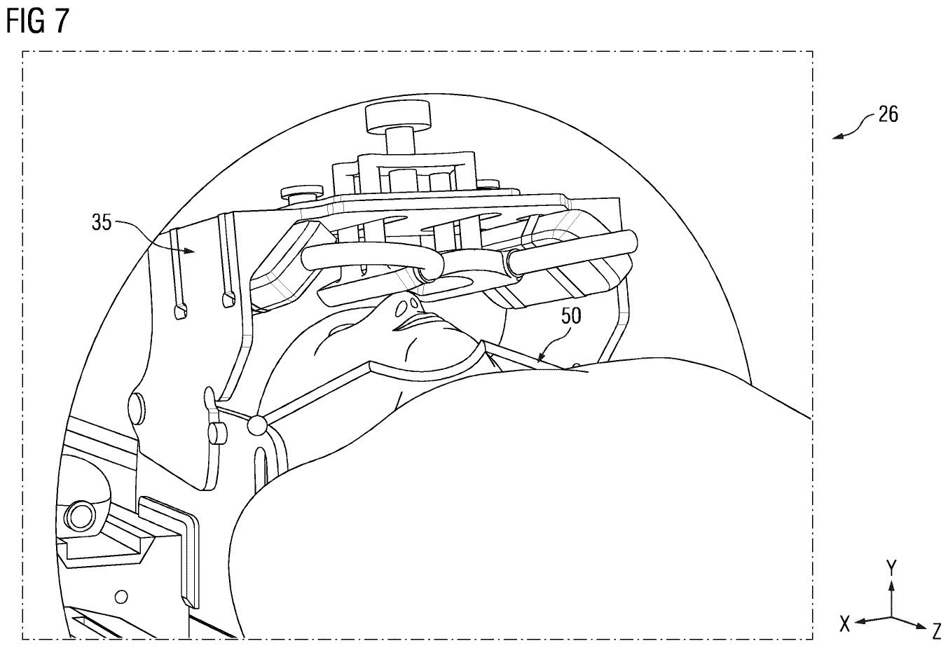

FIG. 7 shows an alternative embodiment variant of a dental coil 26 according to the present invention having a fixing element 50. In the present example, the fixing element 50 comprises an elastic band which encompasses the chin or the lower jaw of the patient 15. The elastic band is preferably mechanically connected to the retainer 35. As a result of the elastic deflection of the elastic band during the positioning of the fixing element 50 on the chin or lower jaw of the patient 15, elastic restoring forces occur in the elastic band which stabilize the head of the patient or fix it in place in a predetermined position relative to the dental coil 26. It is furthermore conceivable that the elastic band of the fixing element 50 is connected to a pull mechanism (not shown) which is embodied to press the elastic band downward with a greater force against the head of the patient 15.

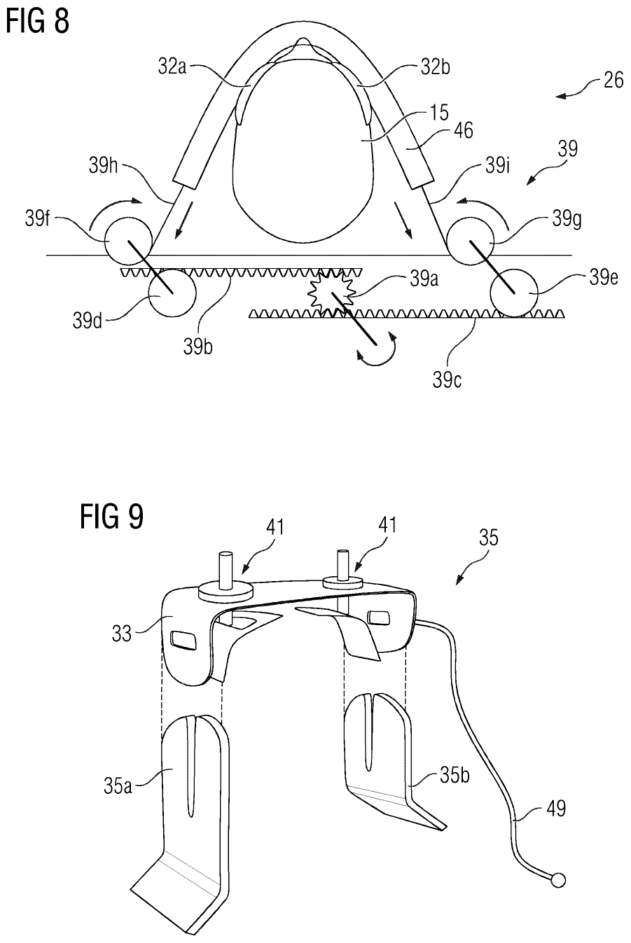

FIG. 8 shows an embodiment variant of the dental coil 26 according to the present invention in which at least the second element 32 is mechanically connected to a flexible holding element 46. The flexible holding element 46 is embodied to shape the second element 32 to conform to the jaw region of the patient 15 and to hold it in place in a position appropriate for use for performing a magnetic resonance measurement of the jaw region 43. In the present example, the flexible holding element 46 comprises an elastic material having a substantially cuboid shape which encloses the jaw region 43 of the patient 15 and shapes the two wings 32a and 32b of the second element 32 to conform to the jaw region 43. The dental coil 26 has a pull mechanism 39 which is mechanically connected to a first end of the flexible holding element 46 on the left cheek region and a second end of the flexible holding element 46 on the right cheek region of the patient 15. The tractive force is provided by actuating, in particular turning, a central gear element 39a. The central gear element 39a is embodied to rotate two gear rods 39b and 39c or to position them relative to each other so that two tension rods 39d and 39e are turned or moved relative to each other. The two tension rods 39d and 39e are each further connected to a spring roller 39f and 39g which keep the tension elements 39h and 39i under tension and/or transmit a tractive force or a direction of the tractive force from the tension rods 39d and 39e onto the holding element 33. The tractive force which is provided by means of the pull mechanism 39 is oriented in such a way that the wings 32a and 32b of the second element 32 are pressed downward by means of the flexible holding element 46 onto the jaw region of the patient 15. The first element 30 can be positioned correspondingly to the second element 32 on the flexible holding element 46 or it can be positioned separately therefrom on the jaw region 43 of the patient 15. It is in particular conceivable that the flexible holding element 46 has a recess or an opening which is designed to receive the first element 30 so that the first element 30 can be positioned independently of the flexible holding element 46 on the jaw region 43 of the patient.

FIG. 9 shows an embodiment variant of a retainer 35 of the dental coil 26 according to the present invention. In the present example, the retainer 35 has two carrier elements 35a and 35b which flank the head of the patient 15 (not shown) when the dental coil 26 is positioned in a manner appropriate for use. The retainer 35 further comprises a holding element 33 which is embodied to hold the first element 30 (not shown) and/or the second element 32 (not shown) in place on the jaw region 43 of the patient 15. The holding element 33 has in particular an adjustment mechanism 41 which is embodied to shape at least the second element 32 (or a third element) to conform to the jaw region 43 of the patient 15. In the example shown, the holding element 33 is particularly easy to release from the retainer 35 in order to allow access for the head of the patient 15 to a volume between the carrier elements 35a and 35b.

In the present example, the holding element 33 is connected to a safety mechanism 49 which is embodied as a ripcord. The ripcord can be embodied to release a mechanical connection between the retainer 35 and the holding element 33 if a predetermined tractive force on the ripcord is exceeded. The holding element 33 can therefore be released from the retainer 35 by means of the ripcord in an emergency. Alternatively, the safety mechanism 49 can also be implemented as a pushbutton, a switch or the like. The safety mechanism 49 can in particular be embodied to control or actuate an actuating element which releases a mechanical connection between the holding element 33 and the retainer 35. For this purpose, the safety mechanism 49 can also comprise a camera and/or a microphone which are embodied to record a gesture and/or a voice message of a person. In this case the gesture and/or the voice message of the person can encode a request to transfer the dental coil 26 into an open position.

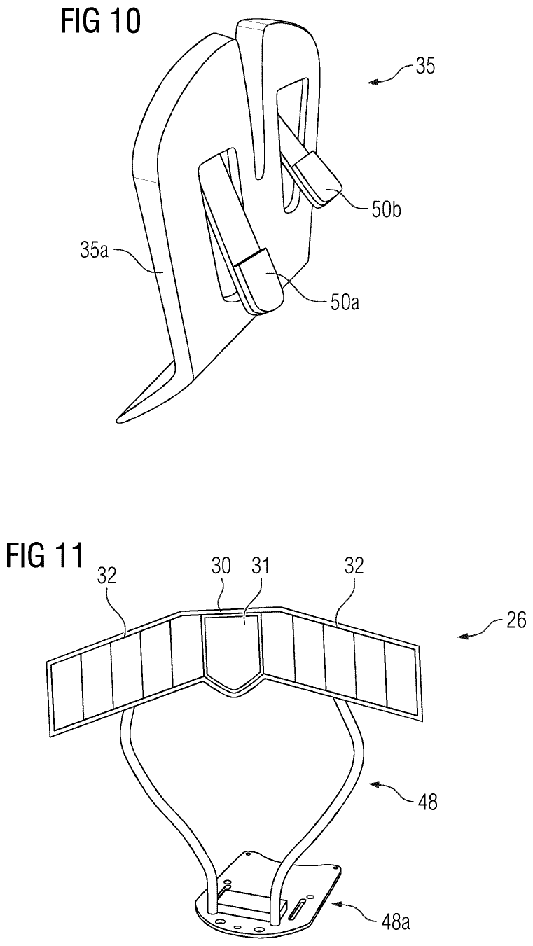

FIG. 10 shows an embodiment variant of a carrier element 35a of the dental coil 26 according to the present invention. In the example shown, the carrier element 35a has two fixing elements 50a and 50b which are pivotably mounted on the carrier element 35a and can be fixed or secured in place in a predetermined position relative to the carrier element 35a. At ends facing in the direction of the patient 15, the fixing elements 50a and 50b have elastic cushions which are positioned laterally on the head of the patient 15 and/or the back of the head of the patient 15 when the dental coil 26 is positioned in a manner appropriate for use. The fixing elements 50a and 50b are embodied in particular to support the head of the patient during a magnetic resonance measurement or to limit or prevent a movement of the head.

FIG. 11 shows an embodiment variant of the dental coil 26 according to the present invention in which the first element 30 and the second element 32 are mechanically connected to a swan-neck mechanism 48. The swan-neck mechanism 48 can comprise a baseplate 48a which is connected to a retainer 35 or a positioning unit 44. In the present case, the first element 30 is mechanically connected to the second element 32. The second element 32 is deformable on account of the flexible element and can be flexed or bent relative to the first element 30 in order to allow it to be shaped to conform to the contour of the jaw region 43 of the patient 15.

Although the present invention has been illustrated and described in more detail on the basis of the preferred exemplary embodiments, the present invention is nonetheless not limited by the disclosed examples and other variations can be derived herefrom by the person skilled in the art without leaving the scope of protection of the present invention.

1. A dental coil comprising:
a first element composed of a dimensionally stable material and including a recess, the recess being configured to receive at least one of a mouth region or a nose region of a patient when the dental coil is positioned for use on a jaw region of the patient; and
a second element having a first flexible element configured to allow the second element to be shaped to conform to a contour of the jaw region of the patient via the first flexible element, wherein
the first element and the second element have an antenna configured to receive radiofrequency signals in a frequency and power range of a magnetic resonance measurement.

2. The dental coil as claimed in claim 1, wherein
the second element is configured to be positioned independently of the first element, or
the first element and the second element are mechanically connected to each other and the second element is held in place by the first element.

3. The dental coil as claimed in claim 1, further comprising:
an adjustment mechanism configured to adjust a shape of the first flexible element to shape the second element to conform to the contour of the jaw region of the patient.

4. The dental coil as claimed in claim 3, wherein the first flexible element has a smart material configured to modify a material property as a function of an excitation signal, wherein the adjustment mechanism is configured to assist in matching of a shape of the second element via transmission of the excitation signal to the first flexible element.

5. The dental coil as claimed in claim 1, further comprising:
a retainer that is mechanically connected to at least one of the first element or the second element, wherein
the at least one of the first element or the second element is configured to be positioned relative to the patient via the retainer, and
the retainer is configured to hold the at least one of the first element or the second element in position appropriate for use on the jaw region of the patient.

6. The dental coil as claimed in claim 5, further comprising:
a positioning unit configured to set at least one of a position or an orientation of the retainer relative to at least one of the patient or a patient support and positioning device.

7. The dental coil as claimed in claim 5, wherein
the retainer includes a pivoting mechanism,
the at least one of the first element or the second element are mounted so as to be pivotable by a maximum angle relative to the patient via the pivoting mechanism when the dental coil is positioned for use on the jaw region of the patient,
the maximum angle of the at least one of the first element or the second element relative to the patient is chosen such that a head of the patient is positionable unobstructed in a position for use relative to the retainer.

8. The dental coil as claimed in claim 5, wherein
the retainer includes a guide mechanism having a guide axis,
the guide axis is aligned substantially parallel to a line of intersection of a sagittal plane and a transverse plane on the jaw region of the patient, and
the guide mechanism is configured to position the first element along the guide axis in order to set a distance between the first element and the jaw region of the patient.

9. The dental coil as claimed in claim 5, wherein the retainer has a swan-neck mechanism or a joint hose.

10. The dental coil as claimed in claim 7, wherein the pivoting mechanism comprises:
a damping element configured to at least one of dampen or limit a movement of the at least one of the first element or the second element when the at least one of the first element or the second element is pivoted along a path of travel.

11. The dental coil as claimed in claim 3, further comprising:
a flexible holding element connected at least to the second element when the dental coil is positioned for use on the jaw region of the patient, wherein
the adjustment mechanism is a pull mechanism that is mechanically connected to the flexible holding element, and
the adjustment mechanism is configured to provide a tractive forces that guides the flexible holding element in a direction of the jaw region of the patient.

12. The dental coil as claimed in claim 1, further comprising:
a third element including a second flexible element, wherein
the second flexible element is configured to allow the third element to be shaped to conform to the contour of the jaw region of the patient,
the third element is arranged on a side of the jaw region of the patient lying opposite the second element when the dental coil is positioned for use, and
the third element has an antenna configured to receive radiofrequency signals in a frequency and power range of a magnetic resonance measurement.

13. The dental coil as claimed in claim 1, further comprising:
a fixing element configured to mechanically connect at least one of a retainer or the first element to a head of the patient in order to fix the head of the patient in place in a position relative to the first element.

14. The dental coil as claimed in claim 1, further comprising:
a safety mechanism configured to transfer the dental coil into an open position when the safety mechanism is actuated by a person in order to remove the dental coil from the position for use on the jaw region of the patient.

15. A magnetic resonance device comprising:
a dental coil as claimed in claim 1, wherein the magnetic resonance device is configured to record radiofrequency signals in a frequency and power range of a magnetic resonance measurement from a jaw region of a patient via the dental coil to perform an imaging scan of the jaw region of the patient.

16. The dental coil as claimed in claim 2, further comprising:
an adjustment mechanism configured to adjust a shape of the first flexible element to shape the second element to conform to the contour of the jaw region of the patient.

17. The dental coil as claimed in claim 4, further comprising:
a retainer that is mechanically connected to at least one of the first element or the second element, wherein
the at least one of the first element or the second element is configured to be positioned relative to the patient via the retainer, and
the retainer is configured to hold the at least one of the first element or the second element in position for use on the jaw region of the patient.

18. The dental coil as claimed in claim 6, wherein
the retainer includes a pivoting mechanism,
the at least one of the first element or the second element are mounted so as to be pivotable by a maximum angle relative to the patient via the pivoting mechanism when the dental coil is positioned for use on the jaw region of the patient,
the maximum angle of the at least one of the first element or the second element relative to the patient is chosen such that a head of the patient is positionable unobstructed in a position for use relative to the retainer.

19. The dental coil as claimed in claim 18, wherein
the retainer includes a guide mechanism having a guide axis,
the guide axis is aligned substantially parallel to a line of intersection of a sagittal plane and a transverse plane on the jaw region of the patient, and
the guide mechanism is configured to position the first element along the guide axis in order to set a distance between the first element and the jaw region of the patient.

20. The dental coil as claimed in claim 7, wherein
the retainer includes a guide mechanism having a guide axis,
the guide axis is aligned substantially parallel to a line of intersection of a sagittal plane and a transverse plane on the jaw region of the patient, and
the guide mechanism is configured to position the first element along the guide axis in order to set a distance between the first element and the jaw region of the patient.
